(19) United States
(12) Patent Application Publication
(10) Pub. No.: US 2022/0284305 A1
(43) Pub. Date: Sep. 8, 2022

(54) BLACK BOX OPTIMIZATION OVER CATEGORICAL VARIABLES

(71) Applicant: International Business Machines Corporation, Armonk, NY (US)

(72) Inventors: Hamid Dadkhahi, Yorktown Heights, NY (US); Karthikeyan Shanmugam, Elmsford, NY (US); Jesus Maria Rios Aliaga, White Plains, NY (US); Payel Das, Yorktown Heights, NY (US)

(21) Appl. No.: 17/189,211

(22) Filed: Mar. 1, 2021

(51) Int. Cl. G06N 5/00; G06N 20/00; G06N 5/04; G16B 40/00; G16B 5/20

(52) U.S. Cl. CPC G06N 5/003; G06N 20/00; G06N 5/04; G16B 40/00; G16B 5/20

(57) ABSTRACT

A black box evaluator is accessed and a surrogate machine learning model that provides estimates for the optimization of categorical values for the black box evaluator is generated, the surrogate machine learning model being based upon observations from previous executions of the black box evaluator. The black box evaluator is optimized by selecting, by an acquisition function executing on a computing device, a new candidate point for the categorical values. The black box evaluator is executed with the new candidate point for the categorical values.

### STATEMENT REGARDING PRIOR DISCLOSURES BY THE INVENTOR OR A JOINT INVENTOR

The following disclosure(s) are submitted under 35 U.S.C. 102(b)(1)(A):
“Fourier Representations for Black-Box Optimization over Categorical Variables,” Hamid Dadkhahi, Karthikeyan Shanmugam, Jesus Rios (Jesus Maria Rios Aliaga), Payel Das, 28 Sep. 2020 (modified: 28 Sep. 2020) ICLR 2021 Conference Blind Submission (OpenReview)—v. 1 abstract only 1 page;
“Fourier Representations for Black-Box Optimization over Categorical Variables,” Hamid Dadkhahi, Karthikeyan Shanmugam, Jesus Rios (Jesus Maria Rios Aliaga), Payel Das, 28 Sep. 2020 (modified: 2 Oct. 2020) ICLR 2021 Conference Blind Submission (OpenReview), v. 2 pages 1-11.
“Fourier Representations for Black-Box Optimization over Categorical Variables,” Hamid Dadkhahi, Karthikeyan Shanmugam, Jesus Rios (Jesus Maria Rios Aliaga), Payel Das, 28 Sep. 2020 (imported: 19 Nov. 2020) ICLR 2021 Conference Blind Submission (OpenReview), v. 3 pages 1-23.
“Fourier Representations for Black-Box Optimization over Categorical Variables,” Hamid Dadkhahi, Karthikeyan Shanmugam, Jesus Rios (Jesus Maria Rios Aliaga), Payel Das, 28 Sep. 2020 (imported: 22 Nov. 2020) ICLR 2021 Conference Blind Submission (OpenReview), v. 4 pages 1-25.
“Fourier Representations for Black-Box Optimization over Categorical Variables,” Hamid Dadkhahi, Karthikeyan Shanmugam, Jesus Rios (Jesus Maria Rios Aliaga), Payel Das, 28 Sep. 2020 (imported: 24 Nov. 2020) ICLR 2021 Conference Blind Submission (OpenReview), v. 5 pages 1-25.
“Fourier Representations for Black-Box Optimization over Categorical Variables,” Hamid Dadkhahi, Karthikeyan Shanmugam, Jesus Rios (Jesus Maria Rios Aliaga), Payel Das, 28 Sep. 2020 (imported: 24 Nov. 2020) ICLR 2021 Conference Blind Submission (OpenReview), v. 6 pages 1-25.
“Fourier Representations for Black-Box Optimization over Categorical Variables,” Hamid Dadkhahi, Karthikeyan Shanmugam, Jesus Rios (Jesus Maria Rios Aliaga), Payel Das, 28 Sep. 2020 (imported: 24 Nov. 2020) ICLR 2021 Conference Blind Submission (OpenReview), v. 7 pages 1-25.

### BACKGROUND

The present invention relates to the electrical, electronic and computer arts, and more specifically, to machine learning and the like.

A plethora of practical optimization problems in machine learning and related fields involve black box functions, with no simple analytical closed forms, that can be evaluated at any arbitrary point in the domain. Optimization of such black box functions poses a unique challenge due to restrictions on the number of possible function evaluations, since evaluating functions of real-world complex processes is often expensive and time consuming. Efficient algorithms for global optimization of expensive black box functions take past queries into account in order to select the next query to the black box function more intelligently.

### SUMMARY

Principles of the invention provide techniques for black box optimization over categorical variables. In one aspect, an exemplary method includes the operations of accessing, by a computing device, a black box evaluator; generating, by the computing device, a surrogate machine learning model that provides estimates for the optimization of categorical values for the black box evaluator, the surrogate machine learning model being based upon observations from previous executions of the black box evaluator; optimizing the black box evaluator by selecting, by an acquisition function 308 executing on the computing device, a new candidate point for the categorical values; and executing, by the computing device, the black box evaluator with the new candidate point for the categorical values.

In one aspect, a computer program product for federated learning comprises a computer readable storage medium having program instructions embodied therewith, the program instructions executable by a computer to cause the computer to: access, by a computing device, a black box evaluator; generate, by the computing device, a surrogate machine learning model that provides estimates for the optimization of categorical values for the black box evaluator, the surrogate machine learning model being based upon observations from previous executions of the black box evaluator; optimize the black box evaluator by selecting, by an acquisition function 308 executing on the computing device, a new candidate point for the categorical values; and execute, by the computing device, the black box evaluator with the new candidate point for the categorical values.

In one aspect, an apparatus comprises a memory and at least one processor, coupled to said memory, and operative to perform operations comprising: accessing, by a computing device, a black box evaluator; generating, by the computing device, a surrogate machine learning model that provides estimates for the optimization of categorical values for the black box evaluator, the surrogate machine learning model being based upon observations from previous executions of the black box evaluator; optimizing the black box evaluator by selecting, by an acquisition function 308 executing on the computing device, a new candidate point for the categorical values; and executing, by the computing device, the black box evaluator with the new candidate point for the categorical values.

As used herein, “facilitating” an action includes performing the action, making the action easier, helping to carry the action out, or causing the action to be performed. Thus, by way of example and not limitation, instructions executing on one processor might facilitate an action carried out by instructions executing on a remote processor, by sending appropriate data or commands to cause or aid the action to be performed. For the avoidance of doubt, where an actor facilitates an action by other than performing the action, the action is nevertheless performed by some entity or combination of entities.

One or more embodiments of the invention or elements thereof can be implemented in the form of a computer program product including a computer readable storage medium with computer usable program code for performing the method steps indicated (a non-transitory computer readable medium comprising computer executable instructions which when executed by a computer cause the computer to perform the method steps disclosed). Furthermore, one or more embodiments of the invention or elements thereof can be implemented in the form of a system (or apparatus) including a memory, and at least one processor that is coupled to the memory and operative to perform exemplary method steps. Yet further, in another aspect, one or more embodiments of the invention or elements thereof can be implemented in the form of means for carrying out one or more of the method steps described herein; the means can include (i) hardware module(s), (ii) software module(s) stored in a computer readable storage medium (or multiple such media) and implemented on a hardware processor, or (iii) a combination of (i) and (ii); any of (i)-(iii) implement the specific techniques set forth herein.

Techniques of the present invention can provide substantial beneficial technical effects. For example, one or more embodiments provide one or more of:

representations for modeling real-valued combinatorial functions over categorical variables;

techniques for learning a surrogate model for the generic Black Box Optimization (BBO) problem;

techniques for optimizing black box functions;

methods for using a version of simulated annealing that utilizes a surrogate model for internal cost-free evaluations before producing the next black box query;

methods for using a version of Monte Carlo tree search (MCTS) in conjunction with a surrogate model as a reward function of the terminal states during intermediate tree traversals/backups in order to improve the sample efficiency of the search algorithm; and

biological (RNA) sequence optimization with competitive or superior performance for the disclosed methods over state-of-the-art counterparts, while substantially reducing the computation time and sample efficiency, respectively.

These and other features and advantages of the present invention will become apparent from the following detailed description of illustrative embodiments thereof, which is to be read in connection with the accompanying drawings.

### BRIEF DESCRIPTION OF THE DRAWINGS

FIG. 1 is a high-level representation of an example learning framework, in accordance with an example embodiment;

FIG. 2A illustrates an RNA structure via Expert-Based Categorical Optimization based on Fourier Representation on Finite Abelian Groups (ECO-G) for n=30, in accordance with an example embodiment;

FIG. 2B illustrates an RNA structure via ECO-G for n=60, in accordance with an example embodiment;

FIG. 2C illustrates an RNA structure via simulated annealing for n=30, in accordance with an example embodiment;

FIG. 2D illustrates an RNA structure via simulated annealing for n=60, in accordance with an example embodiment;

FIG. 2E illustrates a first RNA structure via ECO-G for n=31, in accordance with an example embodiment;

FIG. 2F illustrates a second RNA structure via ECO-G for n=31, in accordance with an example embodiment;

FIG. 3 depicts a cloud computing environment according to an embodiment of the present invention;

FIG. 4 depicts abstraction model layers according to an embodiment of the present invention; and

FIG. 5 depicts a computer system that may be useful in implementing one or more aspects and/or elements of the invention, also representative of a cloud computing node according to an embodiment of the present invention.

### DETAILED DESCRIPTION

Optimization of real-world black box functions defined over purely categorical variables is an active area of research. In general, black box functions, including black box functions that utilize machine learning models, can be computationally expensive to run. Given the teachings herein, the skilled artisan will understand that the disclosed techniques improve the performance of the black box function. In particular, optimization and design of biological sequences with specific functional or structural properties have a profound impact in medicine, materials science, and biotechnology. Standalone acquisition methods, such as simulated annealing (SA) and Monte Carlo tree search (MCTS), are typically used for such optimization problems.

In one example embodiment, in order to improve the performance and sample efficiency of such acquisition methods, existing acquisition methods are used in conjunction with a surrogate model for the black box evaluations over purely categorical variables. To this end, two different representations, a group-theoretic Fourier expansion and an abridged one-hot encoded Boolean Fourier expansion, are utilized. To learn such models, characters of each representation are considered as experts and their respective coefficients are updated via an exponential weight update rule each time the black box is evaluated. Numerical experiments over synthetic benchmarks as well as real-world Ribonucleic Acid (RNA) sequence optimization and design problems demonstrate the representational power of the disclosed methods, which achieve competitive or superior performance compared to state-of-the-art counterparts, while improving the computational cost and/or sample efficiency substantially.

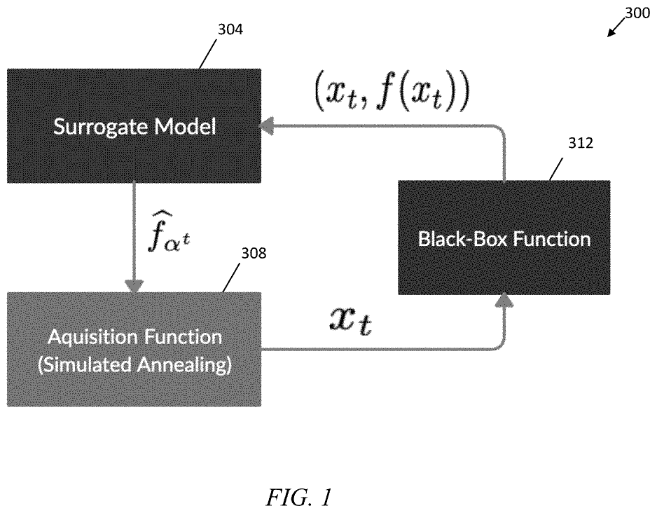

FIG. 1 is a high-level representation of an example learning framework 300, in accordance with an example embodiment. In one example embodiment, a surrogate model 304 provides an estimate for the black box function 312 via observations {(xᵢ, ƒ(xᵢ)):i∈[t]} seen so far. An acquisition function 308 (such as simulated annealing, Monte Carlo tree search, and the like) selects a new candidate point xₜ. The black box function 312 returns the evaluation ƒ(xₜ).

### INTRODUCTION

While black box optimization of real-world functions defined over integer, continuous, and mixed variables has been studied extensively in the literature, limited work has addressed incorporation of purely categorical type input variables. Categorical type variables are particularly challenging when compared to integer or continuous variables, as they do not have a natural ordering. However, many real-world functions are defined over categorical variables. One such problem, which is of wide interest, is the design of optimal chemical or biological (protein, RNA, and Deoxyribonucleic acid (DNA)) molecule sequences, which are constructed using a vocabulary of fixed size, e.g., 4 for DNA/RNA. Designing optimal molecular sequences with improved or novel structures and/or functionalities is pertinent in material science, drug and vaccine design, synthetic biology and many other applications.

Design of optimal sequences is a difficult black box optimization problem over a combinatorially large search space, in which function evaluations often rely on either wet-lab experiments, physics-inspired simulators, or knowledge-based computational algorithms, which are slow and expensive in practice. Another problem of interest is the constrained design problem, e.g., find a sequence given a specific structure (or property), which is inverse of the well-known folding problem. This problem is complex due to the strict structural constraints imposed on the sequence. In fact, one of the ways to represent such a complex structural constraint is to constrain the next choice sequentially based on the sequence elements that have been chosen a priori. Therefore, the black box optimization problem is divided into two settings, depending on the constraint set: (i) Generic Black Box Optimization (BBO) problem, referring to the unconstrained case, and (ii) Design Problem that refers to the case with complex sequential constraints.

Let xₜ be the t-th sequence evaluated by the black box function ƒ. A pertinent question in both settings is the following: Given prior queries x₁, x₂ . . . xₜ and their evaluations ƒ(xᵢ) . . . ƒ(xₜ), how to choose the next query xₜ₊₁? This acquisition should be devised so that over a finite budget of black box evaluations, one is closest to the minimizer in an expected sense over the acquisition randomness.

In the literature, for design problems with sequential constraints, MCTS (Monte Carlo Tree Search)-based acquisitions are often used with real function evaluations ƒ(xₜ). In the generic BBO problems in the unconstrained scenario, Simulated Annealing (SA)-based techniques are typically used as acquisition functions 308. A pertinent missing ingredient in the categorical domain is a surrogate model 304 for the black box evaluations that can interpolate between such evaluations and use cost-free approximate evaluations from the surrogate model 304 internally (in acquisition functions 308) in order to reduce the need for frequently accessing real evaluations. This leads to improved sample efficiency in acquisition functions 308. Due to the lack of efficient interpolators in the categorical domains, existing acquisition functions 308 suffer under a finite budget constraint, due to reliance on only real black box evaluations.

One or more techniques disclosed herein provide one or more substantial beneficial technical effects:

1) Two representations are presented for modeling real-valued combinatorial functions over categorical variables, which are used in order to learn a surrogate model 304 for the generic BBO problem and the design problem. The surrogate model 304 is updated via a hedge algorithm, where the basis functions in the disclosed representations act as experts. The latter update happens once for every real black box evaluation.

2) In the BBO problem, the disclosed method uses a version of simulated annealing that utilizes the current surrogate model 304 for many internal cost-free evaluations before producing the next black box query.

3) In the design problem, the disclosed method uses a version of MCTS in conjunction with the current surrogate model 304 as a reward function of the terminal states during intermediate tree traversals/backups, in order to improve the sample efficiency of the search algorithm.

4) Numerical results, over synthetic benchmarks, as well as real-world biological (RNA) sequence optimization and design problems, demonstrate the competitive or superior performance of the disclosed methods over state-of-the-art counterparts, while substantially reducing the computation time and sample efficiency, respectively. Sample efficiency infers that the black box function 312 is optimized with the minimum number of calls/evaluations of the black box function 312. Sample efficiency effectively reduces computation time since fewer evaluations of a computationally expensive back-box function need to be performed.

Bayesian Optimization (BO) is a commonly used approach for optimization of black box functions 312. However, limited work has addressed incorporation of categorical variables in BO. Early attempts based on converting the black box optimization problem over categorical variables to that of continuous variables have not been very successful. A few BO algorithms have been specifically designed for black box functions 312 over combinatorial domains. In particular, the Bayesian Optimization of Combinatorial Structures (BOCS) algorithm, primarily devised for Boolean functions, employs a sparse monomial representation to model the interactions among different variables, and uses a sparse Bayesian linear regression method to learn the model coefficients. The COMBO algorithm uses Graph Fourier Transform (GFT) over a combinatorial graph, constructed via graph cartesian product of variable subgraphs, to gauge the smoothness of the black box function 312. However, both BOCS and COMBO are hindered by associated high computational complexities, which grow polynomially with both the number of variables and the number of function evaluations. These algorithms work by approximating the black box function 312 with a surrogate; optimizing the surrogate at each step by adding some exploration criteria; and choosing a new point for evaluation from previous optimization. Then the new point is used to update the surrogate.

More recently, a computationally efficient black box optimization algorithm (COMEX) was introduced to address the computational impediments of its Bayesian counterparts. COMEX adopts a Boolean Fourier representation as its surrogate model 304, which is updated via an exponential weight update rule. Nevertheless, COMEX is limited to functions over the Boolean hypercube. COMEX is generalized to handle functions over categorical variables by proposing two representations for modeling functions over categorical variables: an abridged one-hot encoded Boolean Fourier representation and Fourier representation on finite Abelian groups. In one example embodiment, the latter representation is utilized as a surrogate model 304 in combinatorial optimization algorithms. Factorizations based on one-hot encoding has been previously suggested to enable black box optimization algorithms designed for Boolean variables to address problems over categorical variables. The disclosed techniques significantly reduce the number of additional variables introduced upon one-hot encoding, and that such a reduced representation is in fact complete and unique.

For design problems, the RNA sequence design problem (RNA inverse folding) is addressed. One goal is to find an RNA sequence consistent with a given secondary structure, as the functional state of the RNA molecule is determined by the latter structure. Earlier RNA design methods explore the search space by trial and error and use classic cost function minimization approaches such as adaptive random walk, probabilistic sampling, and genetic algorithms. Recent efforts employ more advanced machine learning methods such as different Monte Carlo Tree Search (MCTS) algorithms, e.g., MCTS-RNA or Nested MCTS, and reinforcement learning that either performs a local search or learns complete candidate solutions from scratch. In all these approaches, the assumption is that the algorithm has access to a large number of function evaluations whereas, in one example embodiment, a goal is sample efficiency of each algorithm.

Black Box Optimization Over Categorical Variables

Problem Setting: Given the combinatorial categorical domain X=[k]ⁿ and a constraint set C⊆χ, with n variables each of cardinality k, the objective is to find

x*=argmin_{x∈C}ƒ(x) (1)

where ƒ:χ [symbol] is a real-valued combinatorial function. ƒ is assumed to be a black box function 312, which is computationally expensive to evaluate. As such, one goal is finding x* in as few evaluations as possible. Two variations of the problem are considered depending on how the constraint set C is specified.

Generic BBO Problem: In this case, the constraint set C=χ. For example, the RNA sequence optimization problem that searches for an RNA sequence with a specific property optimized lies within this category. A score for every RNA sequence, reflecting the property to be optimized, is evaluated by a black box function 312.

Design Problem: The constraint set is complex and is only sequentially specified. For every sequence of x₁, x₂ . . . xᵢ including i characters from the alphabet [k], the choice of the next character xᵢ₊₁∈C(x₁, x₂ . . . xᵢ)⊆[k] is specified by a constraint set function C(x₁ . . . xᵢ). The RNA inverse folding problem falls into this category, where the constraints on the RNA sequence are determined by the sequential choices one makes during the sequence design. The goal is to find the sequence that is optimal with respect to a pre-specified structure that also obeys complex sequential constraints.

In order to address this problem, a surrogate model-acquisition-function-based learning framework is adopted, where an estimate for the black box function \hat{ƒ} (i.e., the surrogate model 304) is updated sequentially via black box function evaluations observed until time step t. The selection of candidate points for black box function evaluation is carried out via an acquisition function 308, which uses the surrogate model \hat{ƒ} as an inexpensive proxy (to make many internal calls) for the black box function 312 and produces the next candidate point to be evaluated. The sequence proceeds as follows:

Surrogate model 304 updated on (xₜ,ƒ(xₜ))→Acquisition function 308 makes (many) calls to Surrogate model 304 to propose xₜ₊₁→Surrogate model 304 updated on (xₜ₊₁,ƒ(xₜ₊₁))

In the sequel, two representations are disclosed that can be used as surrogate models 304 for black box combinatorial functions over categorical variables. These representations serve as direct generalizations of the Boolean surrogate model 304 based on Fourier expansion, in the sense that they reduce to the Fourier representation for real-valued Boolean functions when the cardinality of the categorical variables is two. In addition, both approaches can be modified to address the more general case where different variables are of different cardinalities. However, for ease of exposition, all the variables are assumed to be of the same cardinality. Finally, two popular acquisition functions 308 to be used in conjunction with the disclosed surrogate models 304 are introduced in order to propose new queries for subsequent black box function evaluations.

Representations for the Surrogate Model

Two representations for combinatorial functions ƒ:[k]ⁿ [symbol] and an algorithm to update from the black box evaluations are presented. The representations use the Fourier basis in two different ways.

Abridged One-Hot Encoded Boolean Fourier Representation: The one-hot encoding of each variable xᵢ: i∈[n] can be expressed as a (k−1)-tuple (xᵢ₁, xᵢ₂, . . . , xᵢ₍ₖ₋₁₎), where xᵢⱼ∈{−1, 1} are Boolean variables with the constraint that at most one such variable can be equal to −1 for any given xᵢ∈[k].

Consider the following representation for the combinatorial function ƒ:

$${f}_{α} ( x ) = {\sum }_{m = 0}^{n} {\sum }_{ℐ ∈ ( \begin{matrix}[ n ] \\ m\end{matrix} )} {\sum }_{𝒥 ∈ {[ k - 1 ]}^{\vert ℐ \vert}} {α}_{ℐ , 𝒥} {ψ}_{ℐ , 𝒥} ( x ) \qquad ( 2 )$$

In the above, [k−1] [symbol] ^{|} denotes [symbol] -fold cartesian product of the set [k−1]={1, 2, . . . , k−1},

$$( \begin{matrix}[ n ] \\ m\end{matrix} )$$

designates the set or m-sunsets of the set [n], and the monomials [symbol] (x) can be written as

[symbol]

(x)Π_{{(i,j):i=} [symbol] _{,j=} [symbol] _{,} [symbol] _{∈[|} [symbol] _{|]}}xᵢⱼ (3)

A second order approximation (i.e., at m=2) of the representation in (2) can be expanded in the following way:

$${\hat{f}}_{α} ( x ) = {α}_{0} + {\sum }_{i ∈ [ n ]} {\sum }_{ℓ ∈ [ k - 1 ]} {α}_{i ℓ} {x}_{i ℓ} + {\sum }_{( i , j ) ∈ ( \begin{matrix}[ n ] \\ 2\end{matrix} )} {\sum }_{( p , q ) ∈ {[ k - 1 ]}^{2}} {α}_{ijpq} {x}_{ip} {x}_{jq} \qquad ( 4 )$$

### Example: For n=2 variables x₁ and x₂, each of which with cardinality k=3, the one-hot encoding is (x₁₁; x₁₂) and (x₂₁; x₂₂) respectively. From Equation (4), the one-hot encoding factorization for this example can be written as

ƒ(x)=α₀+α₁x₁₁+α₂x₁₂+α₃x₂₁+α₄x₂₂+α₅x₁₁x₂₁+α₆x₁₁x₂₂+α₄x₁₂x₂₁+α₈x₁₂x₂₂

Note that the representation in Equation (2) has far less terms than a vanilla one-hot encoding with all the combinations of one-hot variables included. The reason for this reduction is two-fold: (i) (k−1) Boolean variables model each categorical variable of cardinality k, and, significantly, (ii) each monomial term has at most one Boolean variable xᵢⱼ from its corresponding parent categorical variable xᵢ. The following theorem states that this reduced representation is in fact unique and complete.

Theorem 3.1. The representation in Equation (2) is complete and unique for any real-valued combinatorial function.

Fourier Representation on Finite Abelian Groups: A cyclic group structure [symbol] /kᵢ [symbol] is defined over the elements of each categorical variable xᵢ(i∈[n]), where kᵢ is the cardinality of the latter variable. From the fundamental theorem of abelian groups, there exists an abelian group G which is isomorphic to the direct sum (a.k.a. direct product) of the cyclic groups [symbol] /kᵢ [symbol] corresponding to the n categorical variables:

G≅ [symbol] /k₁ [symbol] ⊕ [symbol] /k₂ [symbol] ⊕ . . . ⊕ [symbol] /kₙ [symbol] (5)

where the latter group includes all vectors (a₁, a₂, . . . aᵢ) such that aᵢ∈ [symbol] /kᵢ [symbol] and ≅ denotes group isomorphism. kᵢ=k(∀i∈[n]) is assumed, for simplicity, but the following representation could be easily generalized to the case of arbitrary cardinalities for different variables.

The Fourier representation of any complex-valued function ƒ(x) on the finite abelian group G is given by

ƒ(x)= [symbol] _{[k]}_{ⁿ} [symbol] [symbol] (x) (6)

where [symbol] are (in general complex) Fourier coefficients, [k]ⁿ is the n-fold cartesian product of the set [k] and [symbol] (x) are complex exponentials (k-th roots of unity) given by

$${ψ}_{ℐ} ( x ) = exp ( \frac{2 π j < x , ℐ >}{k} )$$

Note that, in the general case of different cardinalities for different variables, [symbol] ∈[k₁]×[k₂]× . . . ×[kₙ] where × denotes the cartesian product and the exponent denominator in the complex exponential character is replaced by k=LCM(k₁, k₂, . . . , kₙ). Note that the latter complex exponentials are the characters of the representation, and reduce to the monomials (i.e., in {−1, 1}) when the cardinality of each variable is two. A second order approximation of the representation in (6) can be written as:

$${\hat{f}}_{α} ( x ) = {α}_{0} + {\sum }_{i ∈ [ n ]} {\sum }_{ℓ ∈ [ k - 1 ]} {α}_{i ℓ} exp ( \frac{2 π {jx}_{i} ℓ}{k} ) + {\sum }_{( i , j ) ∈ ( \begin{matrix}[ n ] \\ 2\end{matrix} )} {\sum }_{( p , q ) ∈ {[ k - 1 ]}^{2}} {α}_{ijpq} exp ( \frac{2 π j ( {x}_{i} p + {x}_{i} q )}{k} ) \qquad ( 7 )$$

For a real-valued function ƒ_{α}(x) (which is of interest here), the representation in (6) reduces to

$$\begin{matrix}{f}_{α} ( x ) = ℛ \{ {\sum }_{ℐ ∈ {[ k ]}^{n}} {α}_{ℐ} {ψ}_{ℐ} ( x ) \} \\ = {\sum }_{ℐ ∈ {[ k ]}^{n}} {α}_{r} ℐ {ψ}_{r ,} ℐ ( x ) - {\sum }_{ℐ ∈ {[ k ]}^{n}} {α}_{i} {ℐψ}_{i ,} ℐ ( x )\end{matrix} \\ where \qquad ( 8 )$$

$$\begin{matrix}{ψ}_{r ,} ℐ ( x ) = cos ( \frac{2 π < x , ℐ >}{k} ) and {ψ}_{i ,} ℐ ( x ) \\ = sin ( \frac{2 π < x , ℐ >}{k} )\end{matrix} \qquad ( 9 )$$

It is noted that the number of characters utilized in this representation is almost twice as many as that of monomials used in the previous representation.

Surrogate Model Learning: The learning algorithm of combinatorial optimization is adopted with expert advice in the following way, for example. The monomials [symbol] (x) in (3) and the characters [symbol] (x) in (9) are considered as experts. For each surrogate model 304, a pool of such experts is maintained, the coefficients of which are refreshed sequentially via an exponential weight update rule. The proposed algorithm is referred to as Expert-Based Categorical Optimization (ECO) and the two versions of the algorithm with the two proposed surrogate models 304 are called ECO-F (based on the One-Hot Encoded Boolean Fourier Representation) and ECO-G (based on Fourier Representation on Finite Abelian Groups), respectively. A summary of the algorithm is given below.

Acquisition Functions

Two popular acquisition functions 308, namely Simulated Annealing (SA) and Monte Carlo Tree Search (MCTS), work with a surrogate model 304 and use cost-free evaluations of the surrogate model 304 to select the next query for the black box evaluation. In the literature, SA has been used for the generic BBO problems whereas MCTS has been used for the design problems.

SA as Acquisition Function: The disclosed acquisition function 308 is devised so as to minimize \hat{ƒ}_{α}(x), the current estimate for the surrogate model 304. A simple strategy to minimize \hat{ƒ}_{α}(x) is to iteratively switch each variable into the value that minimizes \hat{ƒ}_{α} given the values of all the remaining variables, until no more changes occur after a sweep through all the variables xᵢ=(∀i∈[n]). A strategy to escape local minima in this context is to allow for occasional increases in \hat{ƒ}_{α} by generating a Markov Chain (MC) sample sequence (for x), whose stationary distribution is proportional to exp

$$( \frac{- {\hat{f}}_{α} ( x )}{s} ) ,$$

where s is gradually reduced to zero. This optimization strategy was first applied in a Simulated Annealing algorithm to solve combinatorial optimization problems. The Gibbs sampler is used to generate such a MC by sampling from the full-conditional distribution of the stationary distribution, which in this case is given by the SoftMax distribution over {−\hat{ƒ}_{α}(xᵢ= [symbol] , x₋ᵢ)/s [symbol] _{∈[k]}, for each variable xᵢ conditional on the values of the remaining variables x₋ᵢ. By decreasing s from a high value to a low one, the MC is allowed to first search at a coarse level avoiding being trapped in local minima.

Algorithm 1 (below) presents an example simulated annealing (SA) version for categorical domains, where s(t) is an annealing schedule, which is a non-increasing function of t. The annealing schedule suggested in Spears (William M. Spears. Simulated annealing for hard satisfiability problems. In DIMACS Workshop: Cliques, Coloring, and Satisfiability, pp. 533-557, 1993) is used, which follows an exponential decay with parameter [symbol] given by

$$s ( t ) = exp ( \frac{- ℓ t}{n} ) .$$

In each step of the algorithm, a variable xᵢ(i∈[n]) is picked uniformly at random, the surrogate model 304 (possibly in parallel) is evaluated k times, once for each categorical value [symbol] ∈[k] for the chosen variable xᵢ given the current values x₋ᵢ for the remaining variables. xᵢ is then updated with the sampled value in [k] from the corresponding SoftMax distribution.

MCTS as Acquisition Function: The design problem is formulated as an undiscounted Markov decision process (S, A, T, R). Each state s∈S corresponds to a partial or full sequence of categorical variables of lengths in [0, n]. The process in each episode starts with an empty sequence s₀, the initial state. Actions are selected from the set of permissible additions to the current state (sequence) sₜ at each time step t, Aₜ=A(sₜ)⊂A. The transition function Tis deterministic, and defines the sequence obtained from the juxtaposition of the current state sₜ with the action aₜ, i.e., sₜ₊₁=T(sₜaₜ)=sₜ∘aₜ. The transitions leading to incomplete sequences yield a reward of zero. Complete sequences are considered as terminal states, from which no further transitions (juxtapositions) can be made. Once the sequence is complete (i.e., at a terminal state), the reward is obtained from the current surrogate reward model \hat{ƒ}_{α}. Thus, the reward function is defined as R(sₜ, aₜ, sₜ₊₁)=−\hat{ƒ}_{α}(sₜ₊₁) if sₜ₊₁ is terminal, and zero otherwise.

MCTS is a popular search algorithm used for design problems. MCTS is a rollout algorithm which keeps track of the value estimates obtained via Monte Carlo simulations in order to progressively make better selections. The Upper Coincidence bounds applied to Trees (UCT) selection criteria, is typically used as tree policy, where action at aₜ state sₜ in the tree search is selected via:

$${π}^{𝒯} ( {s}_{t} ) = arg {max}_{α ∈ A ( {s}_{t} )} Q ( {s}_{t} , a ) + c \sqrt{\frac{ln N ( {s}_{t} )}{N ( {s}_{t} , a )}} ,$$

where [symbol] is the search tree, c is the exploration parameter, Q(s, a) is the state-action value estimate, and N(s) and N(s, a) are the visit count for the parent state node and the candidate state-action edge, respectively. For the selection of actions in states outside the tree search, a random default policy is used: π^{RS}(sₜ)=unif(Aₜ).

A summary of an example algorithm is given in Algorithm 2 (listed below). Starting with an empty sequence s₀ at the root of the tree, the tree policy is followed until a leaf node of the search tree is reached (selection step). At this point, the state corresponding to the leaf node is appended to the tree and a value function estimate for its children (extension step) is initialized. From the reached leaf node, the default policy is followed until a terminal state is reached. At this point, the sequence corresponding to this terminal state is plugged into the surrogate reward model −\hat{ƒ}_{α} and observe the reward r. This reward is backed up from the leaf node to the root of the tree in order to update the value estimates Q(s, a) via Monte Carlo (i.e., using the average reward) for all visited (s, a) pairs along the path. This process is repeated until a stopping criterion (typically a max number of playouts) is met, at which point the sequence s_{best} with maximum reward r_{best} is returned as the output of the algorithm.

Algorithm 1 SA for Categorical Variables with Surrogate Model

| | |
|---|---|
| 1: | Inputs: surrogate model \hat{ƒ}_{α}, annealing schedule s(t), categorical domain X |
| 2: | Initialize x ∈ X |
| 3: | t = 0 |
| 4: | repeat |
| 5: | i~unif([n]) |
| 6: | xᵢ\|x₋ᵢ |
| | $Softmax ( {\{ \frac{- {\hat{f}}_{{α}_{t}} ( {x}_{i} = , {x}_{- i} )}{s ( t )} \}}_{∈ [ k ]} )$ |
| 7: | t ← t + 1 |
| 8: | until Stopping Criteria |
| 9: | return x |

Algorithm 2 MCTS with Surrogate Reward

| | |
|---|---|
| 1: | Inputs: surrogate model \hat{f}_{α}, search tree T |
| 2: | Initialize s_{best} = { }, r_{best} = −∞ |
| 3: | repeat |
| 4: | s_{leaf} ← Selection (π^{T}) |
| 5: | T ← T ∪ {s_{leaf}} |
| 6: | sₜ ← Simulation (π^{RS}, s_{leaf}) |
| 7: | r ← −\hat{f}_{α}(sₜ) |
| 8: | Backup(s_{leaf}, r) |
| 9: | if r > r_{best} then |
| 10: | r_{best} ← r and s_{best} ← sₜ |
| 11: | end if |
| 12: | until Stopping Criteria |
| 13: | return s_{best} |

Computational Complexity: The computational complexity per time step associated with learning the surrogate model 304, for both disclosed representations, is in [symbol] (d)= [symbol] (k^{m-1}n^{m}), and is thus linear in the number of experts d. Moreover, the complexity of the simulated annealing algorithm (Algorithm 1) is in [symbol] (kk^{m-1}n^{m-1}n)= [symbol] (kd), assuming that the number of iterations in each SA run is in [symbol] (n). As a result, the overall complexity of the algorithm is in [symbol] (kd). Finally, the computational complexity of each playout in Algorithm 2 is in [symbol] (kn), leading to an overall complexity of [symbol] (kd), assuming

$$𝒪 ( \frac{d}{n} )$$

playouts per time step.

Experiments and Results

The performance of the proposed representations, when used as surrogate/reward model in conjunction with search algorithms (SA and MCTS) in BBO and design problems, was measured. The learning rate used in exponential weight updates is selected via an anytime learning rate schedule. The maximum degree of interactions used in the surrogate models 304 is set to two for all the problems; increasing the max order improved the results only marginally. The sparsity parameter λ in exponential weight updates is set to 1 in all the experiments. Experimentally, the learning algorithm is fairly insensitive to the variations in the latter parameter. In each experiment, the results were reported averaged over multiple runs (20 runs in BBO experiments and 10 runs in design experiments)±one standard error of the mean. The experiments were run on machines with CPU cores from the Intel Xeon E5-2600 v3 family.

BBO Experiments: The performance of the disclosed ECO algorithms is compared in conjunction with SA with two baselines, random search (RS) and simulated annealing (SA), as well as a state-of-the-art Bayesian combinatorial optimization algorithm (COMBO). In particular, two synthetic benchmarks (Latin square problem and pest control problem) and a real-word sequence design problem in biology, RNA sequence optimization, were considered. In addition to the performance of the algorithms in terms of the best value of ƒ(x) observed until a given time step t, the average computation time per time step of the disclosed algorithm is measured versus that of COMBO. The decay parameter used in the annealing schedule of SA is set to [symbol] =3 in all the experiments. In addition, the number of SA iterations in the disclosed surrogate models 304 is set to T=3×n. Intuitively, each of these parameters creates an exploration-exploitation trade-off. The smaller (larger) the value of [symbol] or T, the more exploratory (exploitative) is the behavior of SA. The selected values seem to create a reasonable balance; tuning these parameters may improve the performance of the acquisition function 308.

Synthetic Benchmarks: Two synthetic problems are considered: Latin square problem, a commonly used combinatorial optimization benchmark, and a pest control problem. In both problems, there are n=25 categorical variables, each of cardinality k=5. A Latin square of order k is a k×k matrix of elements xᵢⱼ∈[k], such that each number appears in each row and column exactly once. When k=5, the problem of finding a Latin square has 161, 280 solutions in a space of dimensionality 5²⁵. The problem of finding a Latin square of order k is formulated as a black box optimization by imposing an additive penalty of one for any repetition of numbers in any row or column. Hence, function evaluations are in the range [0, 2k(k−1)], and a function evaluation of zero corresponds to a Latin square of order k. A noisy version of this problem is considered, where an additive Gaussian noise with zero mean and standard deviation of 0.1 is added to function evaluations observed by each algorithm. Both ECO-F and ECO-G outperform the baselines with a considerable margin. In addition, ECO-G outperforms COMBO until time step t=190. At larger time steps, COMBO outperforms the other algorithms, however, this performance comes at the price of a far larger computation time. ECO-F and ECO-G offer a speed-up over COMBO by a factor of roughly 100 and 50, respectively.

RNA Sequence Optimization Problem: Consider an RNA sequence as a string A=a₁ . . . aₙ of n letters (nucleotides) over the alphabet ∈={A, U, G, C}. A pair of complementary nucleotides aₜ and a₁, where (i<j), can interact with each other and form a base pair (denoted by (i, j)), A-U, C-G and G-U being the energetically stable pairs. Thus, the secondary structure, i.e., the minimum free energy structure, of an RNA can be represented by an ensemble of pairing bases. A number of RNA folding algorithms use a thermodynamic model and dynamic programming to estimate minimum free energy (MFE) of a sequence. However, the O(n³) time complexity of these algorithms prohibits their use for evaluating substantial numbers of RNA sequences and exhaustively searching the space to identify the global free energy minimum, as the number of sequences grows exponentially as 4ⁿ.

The RNA sequence optimization problem is formulated as follows: For a sequence of length n, find the RNA sequence which folds into a secondary structure with the lowest MFE. In the experiments, the initial settings are n=30 and k=4. The popular RNAfold package is used to evaluate the MFE for a given sequence. The goal is to find the lowest MFE sequence by calling the MFE evaluator minimum number of times. Both ECO-F and particularly ECO-G outperform the baselines as well as COMBO by a considerable margin.

RNA Design Experiments: The problem is to find a primary RNA sequence ϕ which folds into a target structure w, given a folding algorithm F. Such target structures can be represented as a sequence of dots (for unpaired bases) and brackets (for paired bases). In the disclosed algorithm, the action sets are defined as follows. For unpaired sites Aₜ={A, G, C, U} and for paired sites Aₜ={GC, CG, AU, UA}. At the beginning of each run of the disclosed algorithm (ECO-F and ECO-G in conjunction with MCTS acquisition), a random permutation for the order of locations to be selected is drawn in each level of the tree. The reward value offered by the environment (i.e., the black box function 312) at any time step t corresponds to the normalized Hamming distance between the target structure ω and the structure yₜ=F(xₜ) of the sequence xₜ found by each algorithm, i.e., d_{H}(w, yₜ).

The performance of the disclosed algorithms is compared against RS as a baseline, where random search is carried out over the given structure (i.e., default policy π^{RS}) rather than over unstructured random sequences. Two state-of-the-art algorithms were included in the experiments: one has an exploration parameter, which was tuned in advance (per sequence); the other has a set of 14 hyper-parameters tuned a priori using training data. Note that the latter training phase as well as the former exploration parameter tuning is offered to the respective algorithms as an advantage, whereas for the disclosed algorithm a global set of heuristic choices for the two hyper-parameters is used, rather than attempting to tune the two hyper-parameters. In particular, the exploration parameter c is set to 0.5 and the number of MCTS playouts at each time step to 30×h, where h is the height of the tree (i.e., number of dots and bracket pairs). The latter heuristic choice is made, since the bigger the tree, the more playouts are needed to explore the space.

It is pointed out that the entire design pipeline in state-of-the-art algorithms typically also includes a local improvement step (as a post-processing step), which is either a rule-based search or an exhaustive search over the mismatched sites. The local improvement step was not included in the conducted experiments, since the main interest was measuring sample efficiency of different algorithms. In other words, the question is the following: given a fixed and finite evaluation budget, which algorithm is able to get closer to the target structure.

In experiments, the disclosed algorithms ECO-F and ECO-G (with MCTS acquisition) are able to significantly improve the performance of MCTS when a limited number of black box evaluations is available. All algorithms outperformed RS as expected.

Description of Algorithms

Surrogate Model Learning Algorithm: Let n and k denote the number of variables and the cardinality of each variable, respectively. The surrogate models 304 used in ECO-F and ECO-G correspond to approximations of the representations given in (2) and (8), respectively, where each approximation is obtained by restricting the maximum order of interactions among variables to m. Each term in the latter surrogate models 304, i.e., monomials [symbol] from (3) in ECO-F and characters [symbol] (β∈{r, i}) from (9) in ECO-G, is considered as an expert, denoted by [symbol] (i∈[d]). The number of such experts in ECO-F is

$$d = {\sum }_{i = 0}^{m} ( \begin{matrix}n \\ i\end{matrix} ) {( k - 1 )}^{i}$$

which coincides with the dimensionality of the space kⁿ when m=n, whereas the number of experts in ECO-G is equal to

$$d = 2 {\sum }_{i = 0}^{m} ( \begin{matrix}n \\ i\end{matrix} ) {( k - 1 )}^{i} - 1.$$

The coefficient of each expert ψᵢ is designated by αᵢ. Since the exponential weights, utilized to update the coefficients αᵢ, are non-negative, two non-negative coefficients α₁⁺ and αᵢ⁻ are maintained, which yield αᵢ=αᵢ⁺−αᵢ⁻.

All the coefficients are initialized with a uniform prior, i.e.,

$${α}_{i}^{γ} = \frac{1}{2 d} ( {∀}_{i} ∈ [ d ] and γ ∈ \{ - , + \} ) .$$

In each time step t, a sample xₜ is drawn via Algorithm 1 with respect to the current estimate for the surrogate model \hat{ƒ}_{α}. The latter sample is then plugged into the black box function 312 to obtain the evaluation ƒ(xₜ). This leads to a mixture loss [symbol] as the difference between the evaluations obtained by the surrogate model 304 and the black box function 312 for query xₜ. Using this mixture loss, the individual loss [symbol] for each expert ψᵢ is computed. Finally, each coefficient in the model is updated via an exponential weight obtained according to its incurred individual loss. This process is repeated until stopping criteria are met

Learning Rate

The anytime learning rate (at time step t) used in Algorithm 3 is given by:

$${η}_{t} = \{ \frac{1}{{e}_{t - 1}} , c \sqrt{\frac{ln ( 2 d )}{{v}_{t - 1}}} \} \qquad ( 11 )$$

Algorithm 3: Expert Categorical Optimization

| | |
|---|---|
| 1: | Inputs: sparsity λ, max model order m |
| 2: | $t ← 0 , ∀ γ ∈ \{ - , + \} ∀ i ∈ [ d ] : {a}_{i , γ}^{t} ← \frac{1}{2 d}$ |
| 3: | repeat |
| 4: | xₜ~\hat{ƒ}_{α}_{^{t}} via Algorithm 1 or Algorithm 2 |
| 5: | Observe ƒ(xₜ) |
| 6: | \hat{f}_{α}_{^{t}}(x) ← Σ_{i∈[d]}(α_{i,+}^{t} − α_{i,-}^{t})ψᵢ (x) |
| 7: | [symbol] ^{t+1} \hat{ƒ}_{α}_{^{t}}(x) − ƒ(xₜ) |
| 8: | for i ∈ [d] and γ ∈ {−, +} do |
| 9: | [symbol] ᵢ^{t+1} ← 2λ [symbol] ^{t+1}ψᵢ(xₜ) |
| 10: | α_{i,γ}^{t+1} ← α_{i,γ}^{t}exp (−γηₜ [symbol] ᵢ^{t+1}) |
| 11: | ${a}_{i , γ}^{t + 1} ← λ \cdot \frac{{a}_{i , γ}^{t + 1}}{{Σ}_{μ ∈ \{ - , + \}} {Σ}_{j ∈ [ d ]} {a}_{j , γ}^{t + 1}}$ |
| 12: | end for |
| 13: | t ← t + 1 |
| 14: | until stopping criteria |
| 15: | return \hat{x} * = argmin_{{x}_{ᵢ}_{:∀i∈[t]}}ƒ(xᵢ) |

Algorithm 4 MCTS with Surrogate Reward Model

| 1: | Inputs: surrogate reward model \hat{ƒ}_{α}, exploration parameter |
|---|---|
| c, search tree [symbol] | |
| 2: | s_{best} = { }, r_{best} = −∞ |
| 3: | repeat |
| 4: | Initialize episode t = 0, sₜ = [ ] |
| 5: | while sₜ ∉ [symbol] do |
| 6: | αₜ ← π [symbol] (sₜ) = |
| | $arg {max}_{a ∈ {A}_{t}} Q ( {s}_{t} , a ) + c \sqrt{\frac{lnN ( {s}_{t} )}{N ( {s}_{t} , a )}}$ |
| 7: | sₜ₊₁ ← T(sₜ, αₜ) = sₜ º αₜ |
| 8: | t ← t + 1 |
| 9: | end while |
| 10: | S_{leaf} = Sₜ |
| 11: | [symbol] ← [symbol] ∪ {sₜ} |
| 12: | ∀αAₜ₊₁:N(sₜ, α) = 0, Q(sₜ, α) = 0 |
| 13: | repeat |
| 14: | αₜ ← π^{RS}(sₜ) |
| 15: | sₜ₊₁ ← T(sₜ, αₜ) = sₜ º αₜ |
| 16: | t ← t + 1 |
| 17: | until sₜ is terminal |
| 18: | r ← −\hat{ƒ_{α}}(st) |
| 19: | s ← s_{leaf} |
| 20: | repeat |
| 21: | N(s, α) ← N(s, α) + 1 |
| 22: | $Q ( s , a ) ← Q ( s , a ) + \frac{1}{N ( s , a )} ( r - Q ( s , a ) )$ |
| 23: | s ← parent(s); α ← visited action on s |
| 24: | until s is the root node |
| 25: | if r > r_{best} then |
| 26: | r_{best} ← r and s_{best} ← sₜ |
| 27: | end if |
| 28: | until Stopping Criteria |
| 29: | return s_{best} |

Experimental Results

Latin Square Problem: A latin square of order k is a k×k matrix of elements x_{i,j}∈[k], such that each number appears in each row and column exactly once. When k=5, the problem of finding a latin square has 161, 280 solutions in a space of dimensionality 5²⁵. The problem of finding a latin square of order k is formulated as a black box function 312 by imposing an additive penalty of one for any repetition of numbers in any row or column. As a result, function evaluations are in the range [0, 2k(k−1)], and a function evaluation of zero corresponds to a latin square of order k. A noisy version of this problem was considered, where an additive Gaussian noise with zero mean and standard deviation of σ=0.1 is added to function evaluations observed by each algorithm.

Both of the disclosed techniques outperform the baselines with a considerable margin. In addition, the disclosed techniques match COMBO's performance closely until time step t=190. At larger time steps, COMBO performs better if a larger computation time is available. The disclosed techniques thus offer a speed-up over COMBO in certain instances.

Pest Control Problem: In the pest control problem, given n stations and k−1 pesticide types, the idea is to maintain the spread of a pest (with minimum cost), which is propagating throughout the stations in an interactive and probabilistic fashion. The k-th category for each variable corresponds to the choice of no pesticide at all. Controlling the spread of the pest is carried out via the choice of the right type of pesticide subject to a penalty proportional to its associated cost.

Despite the fact that COMBO is able to find the minimum in fewer time steps (in ≈200 steps) than ECO-F (in ≈360 steps) on average, ECO-F outperforms COMBO during initial time steps (until t≈180). SA performs competitively, but eventually is unable to find the optimal solution to this problem over the designated 500 steps. Since early mistakes are punished inordinately using ECO-G, the performance of ECO-G may be adversely impacted by the interactive nature of the problem. Early mistakes made by ECO-G can also be attributed to the large number of experts (with noisy coefficients) in its model, which in turn promotes an early exploratory behavior.

RNA Sequence Optimization Problem: Structured RNA molecules play a pertinent role in many biological applications, ranging from control of gene expression to protein translation. The native secondary structure of an RNA molecule is usually the minimum free energy (MFE) structure. Consider an RNA sequence as a string A=a₁ . . . aₙ of n letters (nucleotides) over the alphabet Σ{A, U, G, C}. A pair of complementary nucleotides aᵢ and aⱼ, where (i<j), can interact with each other and form a base pair (denoted by (i, j)), A-U, C-G and G-U being the energetically stable pairs. Thus, the secondary structure of an RNA molecule can be represented by an ensemble of pairing bases.

Finding the most stable RNA sequences has immediate applications in material and biomedical applications. Studies show that by controlling the structure and free energy of an RNA molecule, the translation rate and half-life in a cell may be modulated, which is important in the context of viral RNA. A number of RNA folding algorithms use a thermodynamic model and dynamic programming to estimate MFE of a sequence. However, the O(n³) time complexity of these algorithms prohibits their use for evaluating substantial numbers of RNA sequences and exhaustively searching the space to identify the global free energy minimum, as the number of sequences grows exponentially as 4ⁿ.

Here, the RNA sequence optimization problem is formulated as follows: for a sequence of length n, find the RNA sequence that will fold into the secondary structure with the lowest minimum free energy. In experiments, initially, n is set to 30 and k is set 4. The popular “RNAfold” package is then used to evaluate the MFE for a given sequence. The goal is to find the lowest MFE sequence by calling the MFE evaluator a minimum number of times. Both ECO-F and particularly ECO-G outperform the baselines by a considerable margin.

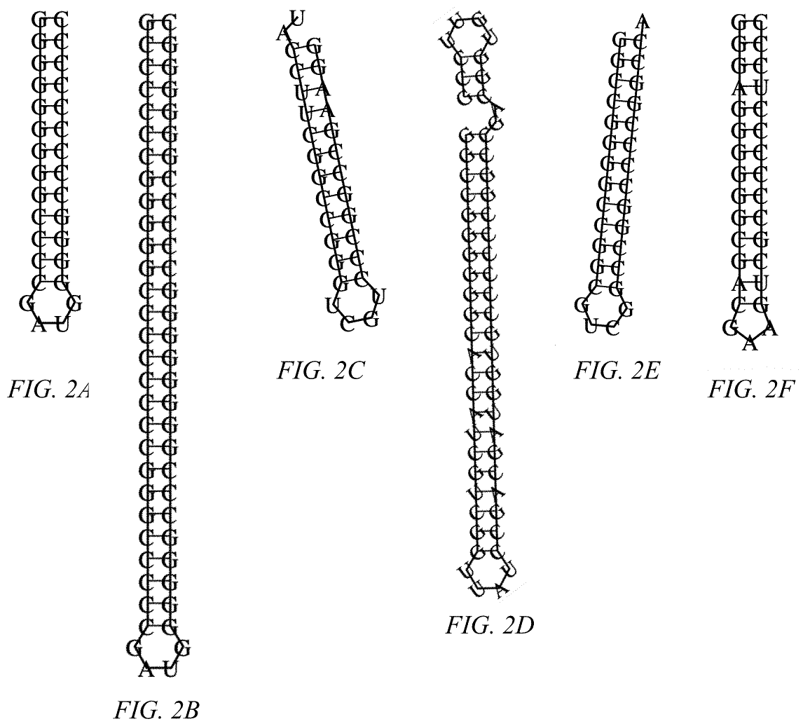

Energy-optimized RNA Structures: Sample RNA sequences obtained via ECO-G after 4000 time steps for n=30 and n=60 are shown in FIGS. 2A and 2B, respectively. The resulting energy optimized sequences (as obtained using the RNAfold service) have high (>90%) GC content that makes the strongest positive contribution to lowering MFE, as pairings between G and C have three hydrogen bonds and are more stable compared to A and U pairings, which have only two. The final single-strand RNA sequence folds into a GC-paired double helix and a 4 nucleotide long hairpin loop in the middle, which is a tetraloop reported in the literature. FIGS. 2E and 2F show two sample structures of the ECO-G optimized sequences for n=31, again showing the same trend. For odd values of n, there is presence of a loop with an odd number of residues or a single unpaired base at the end, but there is still a GC-rich double helix. In contrast, the structures generated by the under-performing algorithms do show presence of unpaired bases and are less in GC content, leading to high-energy structures (e.g., FIGS. 2B and 2C are obtained via SA after 4000 steps for n=30 and 60, respectively).

Given the discussion thus far, it will be appreciated that, in general terms, an exemplary method, according to an aspect of the invention, includes the operations of accessing, by a computing device, a black box evaluator 312; generating, by the computing device, a surrogate machine learning model that provides estimates for the optimization of categorical values for the black box evaluator 312, the surrogate machine learning model being based upon observations from previous executions of the black box evaluator 312; optimizing the black box evaluator 312 by selecting, by an acquisition function 308 executing on the computing device, a new candidate point for the categorical values; and executing, by the computing device, the black box evaluator 312 with the new candidate point for the categorical values.

In one example embodiment, data values are represented using a group-theoretic Fourier expansion, where characters of each representation are considered as experts and respective coefficients of the characters are updated via an exponential weight update rule each time the black box evaluator 312 is executed. In one example embodiment, data values are represented using an abridged one-hot encoded Boolean Fourier expansion, where characters of each representation are considered as experts and respective coefficients of the characters are updated via an exponential weight update rule each time the black box evaluator 312 is executed.

In one example embodiment, a one-hot encoding of each variable xᵢ:i∈[n] is expressed as a (k−1)-tuple (xᵢ₁, xᵢ₂, . . . , xᵢ₍ₖ₋₁₎), where xᵢⱼ∈{−1,1} are Boolean variables with a constraint that at most one such variable is equal to −1 for any given xᵢ∈[k]. In one example embodiment, the black box evaluator 312 is utilized to generate one or more candidate biological molecule sequences that have desirable properties, which are constructed using a vocabulary of fixed size, and synthesizing the optimal biological molecule sequence. In one example embodiment, the black box evaluator 312 is utilized to design optimal sequences over a combinatorially large search space.

In one example embodiment, the black box evaluator 312 is utilized to find a sequence given a specific structure. In one example embodiment, the generation of the surrogate machine learning model is performed via a hedge algorithm where basis functions act as experts. In one example embodiment, Monte Carlo tree search (MCTS) and the surrogate machine learning model are used as a reward function of terminal states during intermediate tree traversals and backups. In one example embodiment, the Monte Carlo tree search (MCTS) utilized is the standard Monte Carlo tree search algorithm (described in detail herein—see, e.g., Algorithm 4). The skilled artisan will appreciate that other types of MCTS techniques may be used in certain embodiments. In one example embodiment, simulated annealing utilizing a surrogate model 304 is performed for internal cost-free evaluations before producing a next black box query. In one example embodiment, the black box evaluator 312 is based on a machine learning model.

In one aspect, a computer program product for federated learning comprises a computer readable storage medium having program instructions embodied therewith, the program instructions executable by a computer to cause the computer to: access, by a computing device, a black box evaluator 312; generate, by the computing device, a surrogate machine learning model that provides estimates for the optimization of categorical values for the black box evaluator 312, the surrogate machine learning model being based upon observations from previous executions of the black box evaluator 312; optimize the black box evaluator 312 by selecting, by an acquisition function 308 executing on the computing device, a new candidate point for the categorical values; and execute, by the computing device, the black box evaluator 312 with the new candidate point for the categorical values.

In one aspect, an apparatus comprises a memory and at least one processor, coupled to said memory, and operative to perform operations comprising: accessing, by a computing device, a black box evaluator 312; generating, by the computing device, a surrogate machine learning model that provides estimates for the optimization of categorical values for the black box evaluator 312, the surrogate machine learning model being based upon observations from previous executions of the black box evaluator 312; optimizing the black box evaluator 312 by selecting, by an acquisition function 308 executing on the computing device, a new candidate point for the categorical values; and executing, by the computing device, the black box evaluator 312 with the new candidate point for the categorical values.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

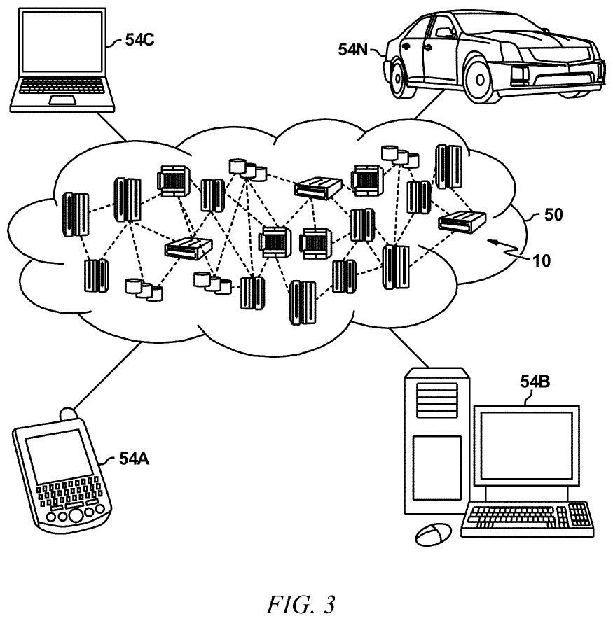

Referring now to FIG. 3, illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 3 are intended to be illustrative only and that computing nodes 10 and cloud computing environment 50 can communicate with any type of computerized device over any type of network and/or network addressable connection (e.g., using a web browser).

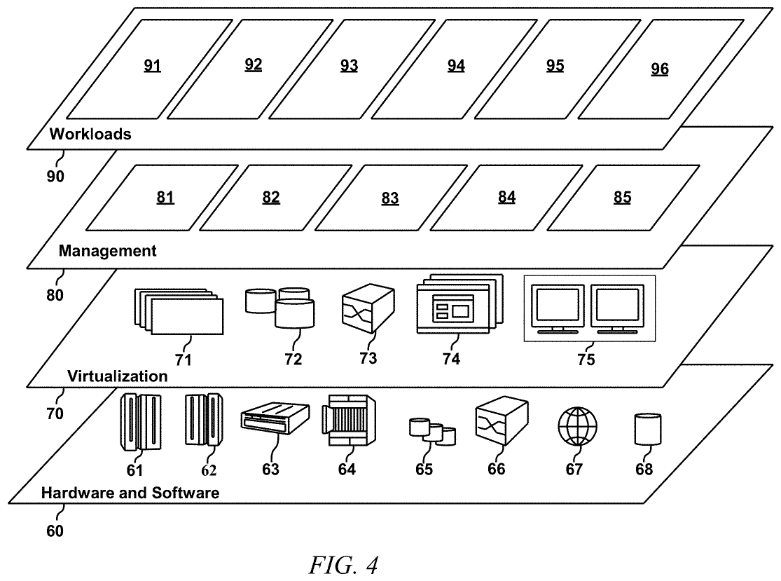

Referring now to FIG. 4, a set of functional abstraction layers provided by cloud computing environment 50 (FIG. 3) is shown. It should be understood in advance that the components, layers, and functions shown in FIG. 4 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and a cloud-based component for black box optimization over categorical variables 96 (it being understood that different embodiments can be cloud-implemented, non-cloud-implemented, or implemented partially in the cloud).

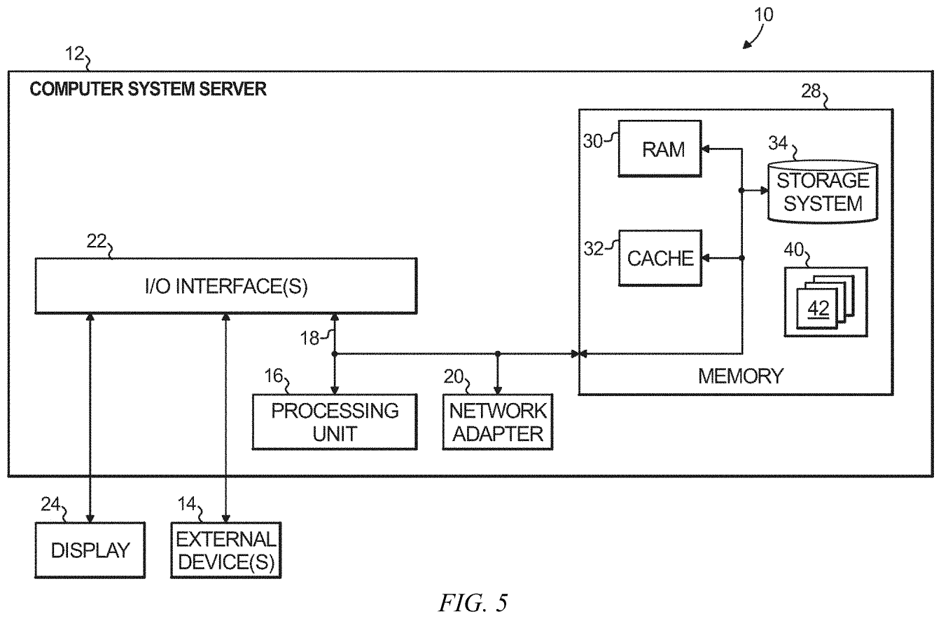

One or more embodiments of the invention, or elements thereof, can be implemented in the form of an apparatus including a memory and at least one processor that is coupled to the memory and operative to perform exemplary method steps. FIG. 5 depicts a computer system that may be useful in implementing one or more aspects and/or elements of the invention, also representative of a cloud computing node according to an embodiment of the present invention. Referring now to FIG. 5, cloud computing node 10 is only one example of a suitable cloud computing node and is not intended to suggest any limitation as to the scope of use or functionality of embodiments of the invention described herein. Regardless, cloud computing node 10 is capable of being implemented and/or performing any of the functionality set forth hereinabove.

In cloud computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 5, computer system/server 12 in cloud computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the invention.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments of the invention as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, and external disk drive arrays, RAID systems, tape drives, and data archival storage systems, etc.

Thus, one or more embodiments can make use of software running on a general purpose computer or workstation. With reference to FIG. 5, such an implementation might employ, for example, a processor 16, a memory 28, and an input/output interface 22 to a display 24 and external device(s) 14 such as a keyboard, a pointing device, or the like. The term “processor” as used herein is intended to include any processing device, such as, for example, one that includes a CPU (central processing unit) and/or other forms of processing circuitry. Further, the term “processor” may refer to more than one individual processor. The term “memory” is intended to include memory associated with a processor or CPU, such as, for example, RAM (random access memory) 30, ROM (read only memory), a fixed memory device (for example, hard drive 34), a removable memory device (for example, diskette), a flash memory and the like. In addition, the phrase “input/output interface” as used herein, is intended to contemplate an interface to, for example, one or more mechanisms for inputting data to the processing unit (for example, mouse), and one or more mechanisms for providing results associated with the processing unit (for example, printer). The processor 16, memory 28, and input/output interface 22 can be interconnected, for example, via bus 18 as part of a data processing unit 12. Suitable interconnections, for example via bus 18, can also be provided to a network interface 20, such as a network card, which can be provided to interface with a computer network, and to a media interface, such as a diskette or CD-ROM drive, which can be provided to interface with suitable media.

Accordingly, computer software including instructions or code for performing the methodologies of the invention, as described herein, may be stored in one or more of the associated memory devices (for example, ROM, fixed or removable memory) and, when ready to be utilized, loaded in part or in whole (for example, into RAM) and implemented by a CPU. Such software could include, but is not limited to, firmware, resident software, microcode, and the like.

A data processing system suitable for storing and/or executing program code will include at least one processor 16 coupled directly or indirectly to memory elements 28 through a system bus 18. The memory elements can include local memory employed during actual implementation of the program code, bulk storage, and cache memories 32 which provide temporary storage of at least some program code in order to reduce the number of times code must be retrieved from bulk storage during implementation.

Input/output or I/O devices (including but not limited to keyboards, displays, pointing devices, and the like) can be coupled to the system either directly or through intervening I/O controllers.

Network adapters 20 may also be coupled to the system to enable the data processing system to become coupled to other data processing systems or remote printers or storage devices through intervening private or public networks. Modems, cable modem and Ethernet cards are just a few of the currently available types of network adapters.

As used herein, including the claims, a “server” includes a physical data processing system (for example, system 12 as shown in FIG. 5) running a server program. It will be understood that such a physical server may or may not include a display and keyboard.

One or more embodiments can be at least partially implemented in the context of a cloud or virtual machine environment, although this is exemplary and non-limiting. Reference is made back to FIGS. 3-4 and accompanying text.

It should be noted that any of the methods described herein can include an additional step of providing a system comprising distinct software modules embodied on a computer readable storage medium; the modules can include, for example, any or all of the appropriate elements depicted in the block diagrams and/or described herein; by way of example and not limitation, any one, some or all of the modules/blocks and or sub-modules/sub-blocks described. The method steps can then be carried out using the distinct software modules and/or sub-modules of the system, as described above, executing on one or more hardware processors such as 16. Further, a computer program product can include a computer-readable storage medium with code adapted to be implemented to carry out one or more method steps described herein, including the provision of the system with the distinct software modules.

One example of user interface that could be employed in some cases is hypertext markup language (HTML) code served out by a server or the like, to a browser of a computing device of a user. The HTML is parsed by the browser on the user's computing device to create a graphical user interface (GUI).

Exemplary System and Article of Manufacture Details

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method comprising:
accessing, by a computing device, a black box evaluator;
generating, by the computing device, a surrogate machine learning model that provides estimates for the optimization of categorical values for the black box evaluator, the surrogate machine learning model being based upon observations from previous executions of the black box evaluator;
optimizing the black box evaluator by selecting, by an acquisition function executing on the computing device, a new candidate point for the categorical values; and
executing, by the computing device, the black box evaluator with the new candidate point for the categorical values.

2. The method of claim 1, wherein data values are represented using a group-theoretic Fourier expansion, where characters of each representation are considered as experts and respective coefficients of the characters are updated via an exponential weight update rule each time the black box evaluator is executed.

3. The method of claim 1, wherein data values are represented using an abridged one-hot encoded Boolean Fourier expansion, where characters of each representation are considered as experts and respective coefficients of the characters are updated via an exponential weight update rule each time the black box evaluator is executed.

4. The method of claim 3, wherein a one-hot encoding of each variable xᵢ:i∈[n] is expressed as a (k−1)-tuple (xᵢ₁, xᵢ₂, . . . , xᵢ₍ₖ₋₁₎), where xᵢⱼ∈{−1,1} are Boolean variables with a constraint that at most one such variable is equal to −1 for any given xᵢ∈[k].

5. The method of claim 1, further comprising utilizing the black box evaluator to generate one or more candidate biological molecule sequences that have desirable properties, which are constructed using a vocabulary of fixed size, and synthesizing the optimal biological molecule sequence.

6. The method of claim 1, further comprising utilizing the black box evaluator to design optimal sequences over a combinatorially large search space.

7. The method of claim 1, further comprising utilizing the black box evaluator to find a sequence given a specific structure.

8. The method of claim 1, wherein the generation of the surrogate machine learning model is performed via a hedge algorithm where basis functions act as experts.

9. The method of claim 1, wherein Monte Carlo tree search (MCTS) and the surrogate machine learning model are used as a reward function of terminal states during intermediate tree traversals and backups.

10. The method of claim 1, further comprising performing simulated annealing utilizing the surrogate machine learning model.

11. The method of claim 10, wherein the simulated annealing utilizing the surrogate machine learning model is performed for internal cost-free evaluations before producing a next black box query.

12. The method of claim 1, wherein the black box evaluator utilizes a black box machine learning model.

13. A computer program product for federated learning, the computer program product comprising a computer readable storage medium having program instructions embodied therewith, the program instructions executable by a computer to cause the computer to:
access, by a computing device, a black box evaluator;
generate, by the computing device, a surrogate machine learning model that provides estimates for the optimization of categorical values for the black box evaluator, the surrogate machine learning model being based upon observations from previous executions of the black box evaluator;
optimize the black box evaluator by selecting, by an acquisition function executing on the computing device, a new candidate point for the categorical values; and
execute, by the computing device, the black box evaluator with the new candidate point for the categorical values.

14. An apparatus comprising:
a memory; and
at least one processor, coupled to said memory, and operative to perform operations comprising:
accessing, by a computing device, a black box evaluator;
generating, by the computing device, a surrogate machine learning model that provides estimates for the optimization of categorical values for the black box evaluator, the surrogate machine learning model being based upon observations from previous executions of the black box evaluator;
optimizing the black box evaluator by selecting, by an acquisition function executing on the computing device, a new candidate point for the categorical values; and
executing, by the computing device, the black box evaluator with the new candidate point for the categorical values.

15. The apparatus of claim 14, wherein data values are represented using a group-theoretic Fourier expansion, where characters of each representation are considered as experts and respective coefficients of the characters are updated via an exponential weight update rule each time the black box evaluator is executed.

16. The apparatus of claim 14, wherein data values are represented using an abridged one-hot encoded Boolean Fourier expansion, where characters of each representation are considered as experts and respective coefficients of the characters are updated via an exponential weight update rule each time the black box evaluator is executed.

17. The apparatus of claim 14, the operations further comprising utilizing the black box evaluator to generate one or more candidate biological molecule sequences that have desirable properties, which are constructed using a vocabulary of fixed size, and synthesizing the optimal biological molecule sequence.

18. The apparatus of claim 14, wherein the generation of the surrogate machine learning model is performed via a hedge algorithm where basis functions act as experts.

19. The apparatus of claim 14, wherein Monte Carlo tree search (MCTS) and the surrogate machine learning model are used as a reward function of terminal states during intermediate tree traversals and backups.

20. The apparatus of claim 14, wherein the operations further comprise performing simulated annealing utilizing the surrogate machine learning model for internal cost-free evaluations before producing a next black box query.
